(19) United States
(12) Patent Application Publication
(10) Pub. No.: US 2005/0084919 A1
(43) Pub. Date: Apr. 21, 2005

(54) Security unit

(76) Inventor: James Redding, Old Coulsdon (GB)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 10/916,016

(22) Filed: Aug. 11, 2004

Related U.S. Application Data
(63) Continuation of application No. 09/913,308, filed on Jan. 17, 2002, now abandoned.

(30) Foreign Application Priority Data
Dec. 2, 1999 (GB) 9903317.7

(51) Int. Cl. C12Q 1/62; B65D 33/16; G06F 17/00

(52) U.S. Cl. 435/10; 235/375

(57) ABSTRACT

Security unit, particularly for storing payments in a retail outlet, comprising a secure, lockable housing with an inlet, a gas feed mechanism for conveying items introduced into the housing, and a removable package for receiving and storing payments. Before the package can be removed, the package is sealed in a tamper evident manner. The package is preferably a plastics container having a neck portion extending from its inlet, and gas outlets remote from the inlet through which gas introduced into the inlet is vented. The feed mechanism preferably comprises a channel through which payments are conveyed and gas inlets provided on opposed sides of the channel, inclined at an angle to that in which the items are conveyed. Gas is jetted through the channels to produce a gas flow which entrains an item to convey this through the channel.

The present invention relates to a security unit or box, and in particular to a security unit for storing securely cash, cheques and/or credit and debit card slips or vouchers. The invention also relates to a feed system for handling items that may be used within such a unit, and to a package, such as a bag, for use within such a unit.

Retail outlets usually have one or more tills provided at sale points on the shop floor. The tills include a till drawer which stores a float from which change may be given to customers. As transactions are completed, the payments tendered by customers are added to the till drawer. Accordingly, the amount of cash together with cheques, credit and debit card slips and coupons will increase in the till drawer. This is a potential security risk as the contents in the till drawer are very vulnerable to theft. This is especially the case as the till drawer is opened for every transaction. Therefore, a thief may attempt to snatch the contents of the till drawer when the drawer is opened. There is also the risk that the till operator will unlawfully remove money from the till drawer.

In an attempt to mitigate this risk, it is known for cash to be removed periodically from the till. This cash is typically removed in unknown quantities.

The cash removed may be taken, for example by a supervisor, from the shop floor to a cash room within the store. This still presents a security risk as there may be a large amount of money in the till drawer at certain times, in particular shortly before the cash is to be removed, and at this time the money is vulnerable to theft as described above. Further, whilst the money is carried from the shop floor, the money is again vulnerable.

In an alternative system, it is known to provide a link from each of the sale points to the cash office by a transportation means, for example an air duct. Money is removed from the till periodically, placed in a canister having dimensions similar to that of the air duct, and the canister is transported through the air duct to the cash room by air pressure in the duct behind the canister. This is an improvement compared to the money being carried by a person over the shop floor as the money is less vulnerable to theft when passing through the air duct since there is no direct access to the duct. However, the problem of a large amount of money being retained in the till where it is vulnerable to theft at certain times is not avoided.

It is therefore known to provide a secure box, typically of metal with a high security lock, at each sale point. The box includes an opening through which notes, cheques and slips can be introduced. The box often includes a separate, removable inner container, for example a glass fibre box, into which the notes or the like are deposited. When the secure box is opened, which may be after the store has closed, the glass fibre box is removed as a single item and taken off the shop floor for processing. In this case, notes may either be introduced to the secure box as they are received or, more usually, a number of notes are introduced into the box when the till becomes too full.

A problem with such known secure boxes of this type is that the amount of money contained in them is not known, and therefore there is still a problem that a member of staff may take money either before this is introduced to the box or after the money has been introduced to the box, and this cannot be detected easily.

EP-A-0,852,279 discloses a security box system in which the removable package is in the form of a bag having an inlet slot for receiving the payments, and which is formed, at least in the region of the inlet slot, from a sealable plastic such that the inlet may be sealed, for example by welding. In a preferred example, the inlet is sealed before the bag is removed from the housing, and preferably the sealing means is associated with a locking means of the housing such that the inlet to the bag is sealed before the lid of the housing can be unlocked. This avoids the problem of the removal of items from the removable package after this has been taken from the secure unit, for example when this is transferred to the store's cash room.

A further problem where the content of the secure box is not known is that the contents of the box must be removed and counted. This introduces a further money-handling step where there is a security risk.

Another problem with conventional money handling systems, including known security boxes, is the conveying or transportation of notes without the risk of jamming or damaging of the notes. Most conventional note handling systems rely either on gravity to cause the notes to drop into a required position, or on a driven system of opposed endless belt conveyors or rollers which nip the notes and drive these to the required position. The problem with a gravity-based system is that it is easy for the notes to become jammed, thereby preventing further notes being added. A driven system using endless belts or rollers is also susceptible to jamming, and may also tear or otherwise damage the notes.

According to a first aspect of the present invention, a security unit comprises a secure, lockable housing including an inlet for items to be stored, a gas feed mechanism for conveying items introduced into the inlet of the housing, and a removable package provided within the housing, the removable package including an inlet through which items conveyed by the gas feed mechanism are introduced into the removable package and a gas outlet through which gas entering the removable package to convey the items to be stored can escape from the package, the unit being arranged so that the inlet of the removable package is sealed in a tamper evident manner before the removable package can be removed from the housing.

With the security unit according to this aspect of the invention, items introduced into the unit through the inlet are stored in a package that is removable allowing the stored items to be removed from the secure unit in a single unit for ease of transportation and processing. As the package cannot be removed until this has been sealed in a tamper evident manner, any attempt to violate the package, for example to remove items contained within the package, will be evident. This improves security. The items to be stored in the removable package within the secure unit are conveyed using a gas feed conveyor system. In this way, items such as, but not limited to, notes, cheques, credit and debit card slips and other papers are conveyed into the housing and package contained therein without significant risk of these jamming or being damaged. The notes or other items do not contact any solid component, and therefore there is reduced risk of these becoming jammed within the channel as compared to a system using endless belts or rollers. Further, as there is no solid, physical means contacting and pulling the items to be conveyed, there is a reduced risk of the item being pulled, stretched or tom. The provision of gas outlets in the removable package allows the gas that entrains and carries the items into the package to leave the package, without over-inflating this. The arrangement according to the present invention therefore provides a secure system for storing payments and the like with improved security and improved handling than the prior art systems.

Preferably the removable package is formed of a plastic material. This has the advantage of being inexpensive to manufacture, and flexible, thereby allowing a reduction in the space taken up by the package when not full compared to prior art solid walled containers. This is of particular advantage for the transportation of the packages after they are removed from the secure units, for example for conveyance to the cash room of a store or for conveyance to a bank or other establishment.

Especially where all or part of the removable package is made of a plastics material, it is preferred that the secure unit includes a heat seal system for heat sealing the removable package. Alternative examples of sealing means include infrared, chemical or vacuum sealers or pressure sensitive adhesive. It is preferred that a sensor is provided to verify that there are no items in the position where the removable package is to be sealed. This is important as there is otherwise the danger that a payment may be damaged by the sealing means, for example by being heated by the heat sealing system, or that the payment will prevent the formation of a proper seal.

It is preferred that the seal includes some form of unique identification, for example a unique identification code, embedded in or printed on the seal. In this way, it will be difficult for the seal to be broken and the package re-sealed without this being evident as it will be difficult to replicate the unique identification.

It is advantageous for the secure unit to include a means to detect the sealing of the removable package before allowing access to the removable package. This makes it possible to ensure that access to the package is prevented whilst this is not in a tamper evident condition, and whilst items could therefore be removed from the package undetected. The secure unit may preferably comprise a lock having a delay such that the access to the interior of the housing, and in particular to the removable package, is prevented for a predetermined period after an attempt is made to open the case, and in particular during this delay, the package may be sealed.

The unit preferably also includes an alarm to alert any attempted, unauthorised opening of the unit. The alarm may include an audio alarm in the unit and/or a remote alarm, for example a signal may be sent to give a visual alert at a control unit. A dye pack may also be provided, for example in or before the feed conveyor. The dye pack is arranged so that, in the event of any tampering with the secure unit, the dye pack explodes so the contents of the secure unit are dyed, making them worthless. This is a further deterrent.

It is preferred that an identification means is provided, for example between the inlet of the housing and the removable package for identifying items that are introduced to the removable package. Such identification of the items are advantageously stored in a memory. If the removable package is identifiable, and is associatable with the stored identification of the items stored in the memory, it is possible to easily determine the content of a particular package. This is particularly advantageous as it means it is not necessary to empty the package and count or confirm its contents. This advantage may also be achieved by provision of a printer that prints details of identified items on the package itself. This avoids a normal money handling operation, which both reduces the amount of time that must be spent processing payments, and also reduces further the risk of unlawful or improper removal of items from the package. In particular, it would allow the package to be removed from the secure unit, and delivered directly to a bank or other institution without any need for the contents to be removed from the package by staff within the store.

The identification may be stored in a chip or other memory device attached to the package. In this case, the memory device may also record details of the content of the package. In this way, the contents of the package can be associated with the package itself.

Where the security unit is used for storing bank notes, a bank note validator is preferably provided for determining the denomination and authenticity of the note before this is passed to and stored in the removable package. If it is determined that the bank note is not authentic, the note is preferably rejected and not introduced into the removable package. Where other items are handled by the secure unit, validators suitable for such items may be provided.

Where the security unit is used for storing items other bank notes, the system may include a printer for printing a unique code on the item before the item is introduced to the security unit. The unique code may include details relating to the item. For example, where the item is a cheque, the code may include the value of the cheque, the transaction number to which the cheque relates, the time and date on which the cheque was received and the sort code. The identification means may comprise an optical character reader that identifies details from the item, or a decoder for reading the code printed on the item.

Beneficially, the unit comprises a means to detect the presence and/or correct fitting of a removable package before allowing items to be stored.

According to a second aspect of the present invention, a conveying system comprises a channel through which items are to be conveyed and a pair of gas inlets provided on opposite sides of the channel, inclined to the angle at which the items are to be conveyed, such that gas from outside the channel is jetted through the inlets to produce a gas flow through the channel which entrains an item to convey this through the channel.

With the conveying system according to the present invention, items such as, but not limited to, notes, cheques, credit and debit card slips and other papers are conveyed through a channel without significant risk of these jamming or being damaged. The notes or other items do not contact any solid component of the channel, and therefore there is reduced risk of these becoming jammed within the channel as compared to a system using endless belts or rollers. Further, as there is no solid, physical means contacting and pulling the items to be conveyed, there is a reduced risk of the item being pulled, stretched or torn.

It is preferred that the gas inlets are in the form of pipes or tubes provided at opposite sides of the inlet end of the channel and angled towards a central plane of the channel. In this case, the gas flows from the inlets will converge towards the centre of the channel, and then flow through the channel. When an item is presented to the channel, this will be entrained by the flow of gas through the channel, and conveyed through the channel. The gas may be supplied by fans driving gas through the pipes or tubes.

To help maintain the conveyed items flat, it is preferred that the gas jets include components directed towards the large upper and lower surfaces of the item. In this case, it is preferred that the inlets are directed into the channel from above and below the point where the item to be conveyed is received in the channel.

Alternatively or additionally to the provision of gas inlets at the inlet end of the channel, inlets may be provided along the length of the channel. In this case, the inlets may be in the form of an array of holes, for example circular or oval holes, much like a cheese grater. The gas will jet through these holes to convey the item. Alternatively, the openings may be in the form of elongate slots extending substantially across the width of the channel. An associated fluid guide or baffle may be provided to direct the jet of gas into the channel. The advantage of slots is that the area through which the gas enters the channel is larger, giving a greater volume of gas to convey articles through the channel. Nevertheless, the resulting velocity of the gas flow will be lower compared to that where holes are used. In either case, the channel may be provided within a region of high pressure, such that gas from the high pressure region flows through the openings into the channel.

The use of air as the gas is advantageous as this may be provided to the channel merely by providing a fan or blower to blow the ambient air without requiring a separate gas supply. Alternatively, a separate source of pressurised gas may be provided.

Where the inlets comprise openings formed in the walls of the channel, these are preferably angled with respect to the longitudinal plane passing through the channel and to the plane normal to the direction through the channel. In this way, a region of high gas pressure can be provided around the channel, and the gas will jet into the channel to give components of fluid flow extending generally normal to the opposed faces of the channel, and components passing longitudinally through the channel. In this way, the item to be conveyed will be suspended between the opposed, typically upper and lower, faces of the channel by the generally normal component of the fluid entering the channel through the openings. This keeps the items flat and spaced from the walls of the channel to prevent jamming or damage.

The openings along the wall of the channel are beneficially formed through a dimple in the surface of the channel. In this case, the dimple may be formed either into or out of the channel. Where the dimple is formed into the channel, the dimple is formed behind the hole in the direction in which the item is to be conveyed. Where the dimple is formed out of the channel, the dimple is formed in front of the hole in the direction in which the item is to be conveyed.

In any case, the channel preferably has a generally oval cross-sectional channel in a plane generally perpendicular to that along which the items are conveyed. This shape is found to give improved fluid flow characteristics and therefore improved conveying properties compared to a rectangular channel.

The air inlets are advantageously formed at an angle of about 45° to the channel. In this way, the component of the fluid flow flowing normal to the opposed faces of the channel is between about 10 and 20%, more preferably between 10 and 15% of the overall fluid flow and the remaining component of the fluid flow is in the direction in which the items are to be conveyed.

The channel may be made of sheet material, such as metal. Alternatively, the channel may be moulded. This is a particularly suitable method of manufacture where the channel is made from a plastics material.

Preferably, a sensor is provided to detect the presence of an item in the channel. In this case, a control means is advantageously provided to control the flow of gas dependent upon the determination of the presence of an item to be conveyed. In particular, it is preferred that there is a nominal gas flow through the channel when no item is detected in the channel and, when an item is detected in the channel, the gas flow is increased to convey the item. This makes it easier to introduce an item to the channel, as there is no, or minimal, gas flow affecting the insertion of the item. Further, the energy requirements of the system are reduced by only supplying significant gas flows when required.

It is preferred that the gas feed system used in the secure unit according to the first aspect of the present invention is in accordance with the second aspect of the present invention.

According to a third aspect of the present invention, a tamper evident package comprises a plastics container having an inlet opening for receiving items to be collected, a neck portion extending from the inlet opening, and a number of gas outlets remote from the inlet portion through which gas introduced into the inlet opening is vented.

The tamper evident package according to the third aspect of the present invention is preferably used in a secure unit in accordance with the first aspect of the invention, and especially one which includes a gas conveying means in accordance with the second aspect of the present invention. The gas outlet allows the gas conveying items into the package to leave the package. If such an outlet is not provided, the gas will inflate the removable package, and prevent the flow of gas into the package, thereby preventing the addition of further items.

The gas outlet is preferably in the form of holes provided in a wall of the package, and more preferable on the side wall of the package opposite the inlet. The number and size of the gas outlets depends upon the gas flow into the package, but advantageously at least 400 holes are provided. Each hole should be sufficiently small that items cannot be removed from the package through the holes. Typically, the holes should be about 2 mm in diameter.

It is preferred that the gas outlet is formed towards the upper part of the package remote from the inlet to the package. In this way, the optimum gas flow into and through the removable package is ensured for depositing items within the package.

An example of the present invention will be described in accordance with the accompanying drawings, in which:

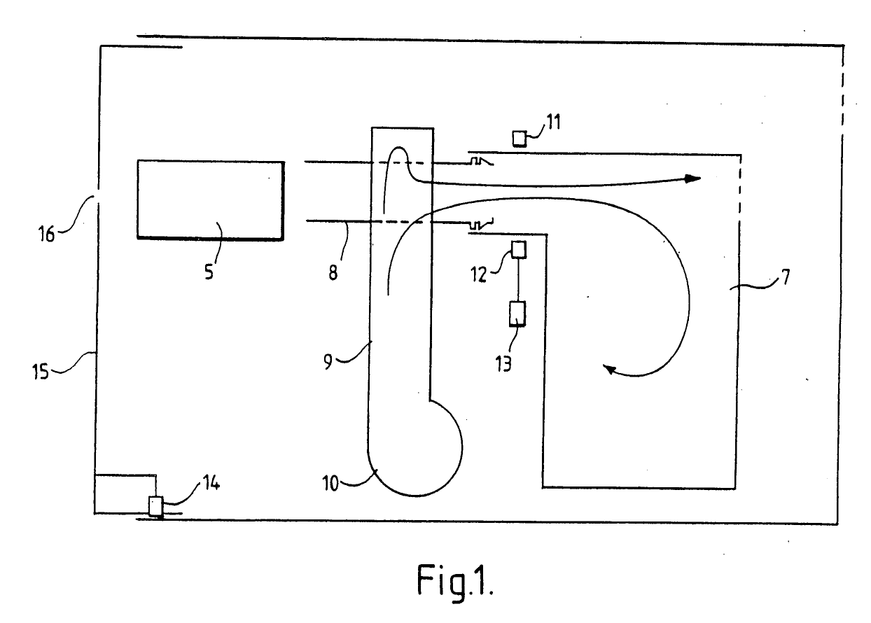

FIG. 1 shows a cross-section through a security unit;

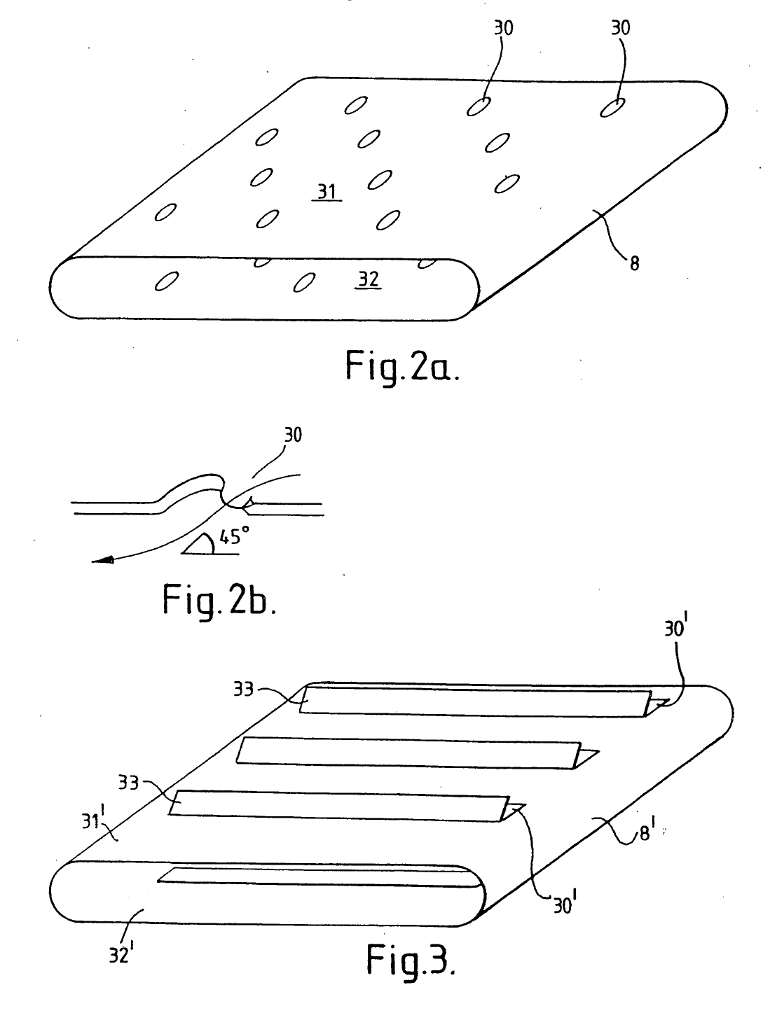

FIG. 2a shows an air-channel conveyor;

FIG. 2b shows a cross-section through an opening in the air-channel conveyor;

FIG. 3 shows an alternative air-channel conveyor;

FIG. 4 shows an air supply system for the air channel;

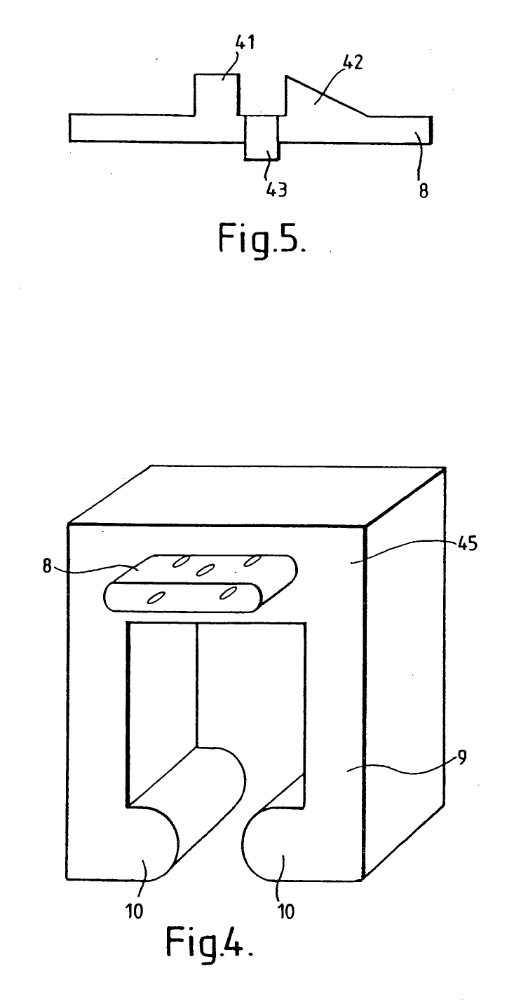

FIG. 5 shows a system for mounting a bag on the air channel;

FIG. 6 shows a first example of a bag;

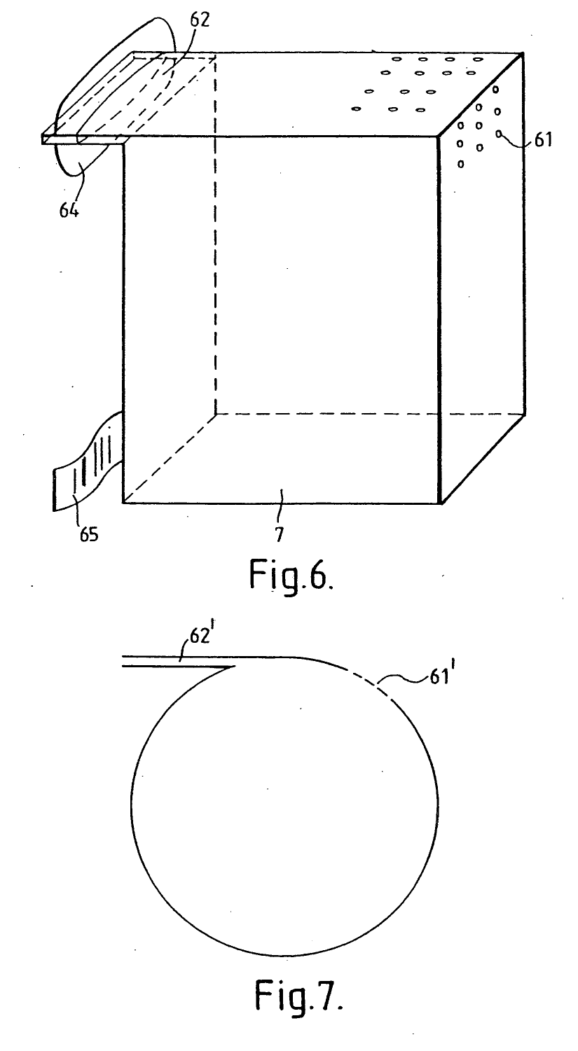

FIG. 7 shows a second example of a bag; and,

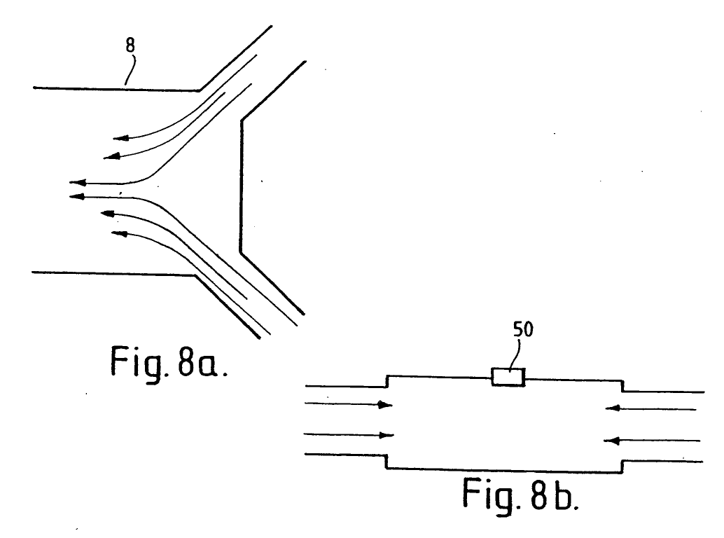

FIG. 8 shows a further example of an air channel conveyor.

The aspects of the present invention will be described with respect to a security unit which forms one example of the first aspect of the present invention, and which comprises a number of individual components, each of which may form an aspect of the invention in their own right. In particular, the unit includes a conveyor system for conveying items. The conveyor system constitutes an example of the second aspect of the present invention. The unit is also described as being provided with a bag that constitutes an example of the third aspect of the present invention. The invention also lies in the combination of two or more of the individual components of the security unit.

The security unit of the present invention will be described initially with reference to one of its preferred functions as a note handling unit, although it is understood that the unit may have other functions for different items.

The unit comprises a housing formed as a lockable metal, typically steel, case. The front 15 of the case includes a chassis on which the components of the unit are mounted. The front 15 is provided on arms (not shown) including rollers to allow the front 15 and the chassis to be removed easily to allow access to the interior of the case. The construction of the case is such that it is secure, so that any attempt to breach the case is resisted due to the strength of the case, and any attempt to breach the case is easily apparent. An audible alarm is provided to warn of any unauthorised attempt to gain access to the unit. The case is locked by an electronic lock 14 that allows the case to be opened only after predetermined procedures have been completed as described below. The security case includes an inlet slot 16 through which notes are inserted, and a note validator 5 that receives and validates the inserted notes. The case includes a void which receives a plastics bag 7 or other package for the notes. Notes from the validator 5 are deposited in the package. When the case is opened, the bag 7 and its contents are removable from the case.

The authenticity and denomination of each note is determined by the validator 5. The validator 5 may be a conventional validator 5 such as the IDS validator available from Global Payment Technologies, Inc. A note is inserted into the validator 5, and is driven by a series of endless belt conveyors and/or rollers, past one or more note recognition devices. The validator determines parameters of the note, for example its size, colour and magnetic properties. These determined parameters are compared with predetermined acceptable ranges of parameters for notes of different denominations. Based on this comparison, the note is either rejected if it cannot be determined to be a genuine note, or accepted and its value determined. Where a note is rejected, the belts or rollers are driven in reverse to eject the note.

All accepted notes that pass through the validator 5 extend into an air channel 8, this being an example of the second aspect of the present invention. One example of an air channel is shown in FIG. 2a. The air channel 8 comprises a generally oval channel having a width of about 90 mm, a height of about 20 mm and a length of about 95 mm. The upper and lower faces of the air channel 8 include an array of openings 30 which are spaced in rows extending across the width of the channel in which adjacent openings 30 are spaced by 20 mm, and with adjacent rows being spaced by 10 mm with a 10 mm offset between openings 30 in adjacent rows. As shown in cross-section in FIG. 2b, each opening 30 is formed by providing a 5 mm hole though a dimple formed in the surface of the channel, the hole being formed at an angle of about 45° to the channel. The resulting openings 30 are similar to those of a cheese grater. The dimple may be formed either into the channel 8 or out of the channel 8. In the case where the dimple is formed into the channel 8, the dimple is formed behind the hole in the direction in which the note is to pass from the validator 5. In the case where the dimple is formed out of the channel 8, the dimple is formed in front of the hole in the direction in which the note is to pass from the validator 5. As shown in FIG. 2b, the dimple causes air from outside the channel 8 to flow into the channel as a laminar air flow having a major component in the direction through the channel 8.

An alternative air channel 8′ is shown in FIG. 3. In this case, the openings comprise elongate slots 30′ provided in the upper and lower faces 31′,32′ of the air channel 8′. A guide 33, which may be pressed out of the channel 8′ to form the elongate slots 30′, directs air into the channel 8′ as a laminar flow generally as described above. In the following, it will be understood that either air channel 8 or 8′ may be used.

As shown in FIG. 4, the channel 8, or the alternative air channel 8′ is provided within a duct 9, both ends of which open to a fan or blower 10, for example a RL90-18/12NG DC radial blower available from PAPST which is able to provide a flow rate of 40 m³h⁻¹. The duct 9 extending from each of the blowers 10 has a width of about 25 mm and a depth of about 70 mm giving a cross-sectional area of about 1750 mm². The ducts 9 from the blowers 10 are linked by a further duct 45 having a height of around 70 mm and a depth of around 70 mm. The air channel 8,8′ is provided within this duct 45. The blowers 10 blow air into the ducts 9, 45, increasing the air pressure on the outside of the channel 8,8′. The high pressure air, typically of around 2 atmospheres, passes through the openings 30 of the channel 8,8′ into the channel 8,8′. Due to the angle at which the openings 30 are formed, and the dimple, the turbulent air in the duct around the channel 8,8′ is directed as a laminar flow into the channel 8,8′ in the direction generally away from the validator 5. As the openings 30 are formed in both the upper and lower faces 31,32 of the channel 8,8′, there will be a component of air passing vertically upwardly and downwardly to the centre of the channel 8,8′. This acts to maintain the note hovering near the middle of the channel 8,8′ so the note can pass freely through the channel 8,8′ without catching on the surfaces of the channel 8,8′. Typically, 10 to 20% of the air flows vertically. The horizontal component of the air, the remaining 80 to 90%, facts to entrain the note and draws this from the validator 5 into the bag 7.

An alternative example of an air feed system is shown in FIGS. 8a and 8b. FIG. 8a shows the feed system in plan view, where items to be conveyed through a channel are conveyed from an inlet end on the right hand side towards the outlet on the left hand side. In the end view of FIG. 8b, shown from the inlet end, items are conveyed away from the viewer. In the case of notes or similar items, these are generally horizontal, namely parallel to the top and bottom surfaces of the channel.

With the arrangement shown in FIG. 8, the air is directed into the channel from either side at an angle of about 45°. This results in the two air flows from either side converging at a central point across the width of the channel, and close to the inlet end, with the combined air flowing longitudinally through the channel, as shown best in the plan view of FIG. 8a. As shown in FIG. 8b, the air flows across the full height of the channel. This consistency throughout the height of the channel ensures that items conveyed through the channel are not bent.

In operation, one or more sensors 50 are provided in the channel to detect the presence of an item to be conveyed, and the direction in which the item is travelling. When no item is detected, the velocity of air flowing through the channel is reduced to a nominal level, for example by decreasing the driving voltage to an air fan supplying the air. This is advantageous both as it reduces the energy consumption of the system and the noise level of the system when nothing is to be conveyed, and as there is minimal air flow through the channel which may affect the insertion of an item to be conveyed. When an item is introduced into the inlet end of the channel, and this is detected by the sensor 50, the velocity of the air supplied to the channel is increased. In this case, the resulting air flow through the channel will entrain the item, and convey this through the channel. When the sensor 50 detects that the item has been conveyed, or after a predetermined period after the presence of the note was detected, the air flow through the channel will again be reduced.

On the outer surface of the air channel 8 at the end remote from the validator 5, there are provided two projections 41,42, the projection 42 nearest the end of the channel 8 remote from the validator 5 having an inclined surface. A sensor element 43 is provided between the two projections 41,42. This is shown in FIG. 5. The projections 41,42 and sensor 43 are provided for location of a bag 7 on the air channel 8 and for ensuring the bag 7 is mounted correctly as described below.

As shown in FIG. 6, the removable package comprises a generally cuboid bag 7 formed of polythene or other plastics material. The bag 7 is an example of the third aspect of the present invention. In one example, the bag 7 has a width of about 120 mm, a length of about 250 mm and a height of about 300 mm. Such a bag 7 has a capacity of about 500 to 700 bank notes. An opening slit is provided across the upper front edge of the bag 7, and a neck 62 of about 50 mm is provided from this opening. An array of holes 61 is provided on the top face of the bag 7 towards the back, and on the rear of the bag 7 towards the top. Typically, at least 100 holes 61 are provided on each of the upper and rear faces of the bag 7, each hole having a diameter of about 2 mm. The bag 7 is designed so that, once inflated, it assumes its generally cuboid shape, although can be flat packed for storage. In another example, the bag 7 has a height of about 360 mm, a length of about 320 mm and a width of about 160 mm, and has a total of around 400 air outlet holes. The case also includes air vent holes through which air can enter the case to be driven by the blowers 10, and from which air leaves the case after leaving the bag 7. The bag 7 includes a plastics rim 64 around the neck 62 of the bag 7 for fitting the bag 7 to an air channel 8 as described below. In an alternative arrangement, an internal removable ring may be provided to be secured to a bag 7 and fitted to the air channel 8.

An alternative bag design is shown in FIG. 7. This bag is a cylindrical bag, again the an upper slit type opening with an associated neck 62′ and air holes 61′ provided in the upper rear of the bag 7.

The notes entrained in the air flow through the air channel 8 are transported by the air stream through the neck 62 of the bag and into the bag. As the air flow enters the large volume in the bag 7, turbulence is introduced into the generally laminar air flow from the channel 8. This turbulence causes the entrained notes to be forced towards the bottom of the bag 7. The turbulent air flow also acts to compress the notes already deposited in the bag 7 to maximise the number of notes which the bag 7 may contain. The air then leaves the bag 7 through the holes 61 in the top and rear of the bag 7. It is important that the velocity of the air flow into the bag 7 is not too great, since this will cause a reduction in the pressure in the top part of the bag 7, and this will cause the notes in the bag 7 to lift.

The bag 7 includes a unique bar code identification mark (not shown) that is embedded in the plastics material from which it is formed. An identical bar code is embedded in a tear off strip 65 that is provided on the bag 7. This strip may be removed from the bag 7 as this is fitted and used for identification. Alternatively, the secure unit may include a thermal printer to print a unique identification on the bag whilst this is provided in the secure unit. In this case, the printer may print not only an identification of the container, but alternatively or additionally may print one or more of the date or time when the container was provided in the secure unit or removed from the secure unit, or the content of the container.

In an alternative embodiment, the bag 7 is provided with a memory chip which can be connected to an interface for identifying the bag. Information relating to the content of the bag, for example from the validator 5, can be written to and stored in the memory chip. This gives a record of the bag's content associated with the bag. Any attempt to remove the chip will be evident.

In use, the bag 7 is provided within the secure case by placing the bag 7 within the void in the rear section of the case, and mounting the open neck 62 of the bag 7 over the end of the air channel 8 remote from the validator 5. The neck 62 of the bag 7 is provided over the channel 8 to create an air right seal. It is important to ensure that a bag 7 is in position and correctly fitted before items are introduced. As shown in FIG. 5, the end of the air channel 8 remote from the validator 5 includes a pair of spaced projections 41,42. The projection 42 near the end of the channel 8 includes an inclined surface. In between the projections there is provided a sensor 43. As described above, the neck 62 of the bag 7 is provided with a solid member 64 which is slid over the sloped surface of the projection 42 nearest the end of the channel 8, and locates between the two projections 41,42. In this position, the presence of the solid member 64 is detected by the sensor 43 and it is determined that the bag 7 is correctly loaded. In the event that the sensor 43 fails to determine that the bag 7 is correctly loaded, the secure unit is disabled to prevent notes being inserted, since otherwise any notes inserted will not be stored in a tamper evident package. Further, an optical sensor may be provided to confirm a bag 7 is provided within the case, and that the sensor 43 has not been unlawfully disabled. A switch is then actuated, causing the fans to be activated and air to flow into the air channel 8 and into the bag 7. This air inflates the bag 7, causing the bag 7 to assume its cuboid arrangement. The dimensions of the bag 7 are slightly greater than the space within the case of the secure unit in which the bag 7 is provided, thereby ensuring that the bag 7 assumes the shape of the space in the case. To further ensure the bag 7 is correctly loaded, the air flow or fan driving the air can be monitored. In the event that a bag 7 is not fitted correctly, or the bag 7 is damaged, for example if this is torn, the air flow will deviate from an expected level, and the error can be detected and the unit disabled. Detection of the bag in this way may be achieved by determination of the drive current of the air fan.

When the bag 7 is loaded, the identity of the bag 7 is recorded in a central unit so the payments deposited in the bag 7 may be identified. To achieve this, a bar code reader associated with the money handling unit reads the bar code embedded in the bag 7, or the bar code on the tear-off strip. In one example, a hand-held bar code reader is provided with the station, and the information is read form the embedded bar code by the reader and this information is transmitted to a memory unit in which the identified contents of the bag 7 are logged. In another example, the validator 5 doubles as a bar code reader. In this case, the tear-off strip from the bag 7 is read through the validator 5 which reads the bar code to identify the bag 7 and this information is transmitted to the memory unit. In a still further example, the bag 7 identification may be manually input to the memory unit. In any case, it is arranged that the validator 5 will not accept notes until the bag 7 has been identified to a central control unit.

As shown in FIG. 1, a heat seal unit is provided to seal the open neck 62 of the bag 7 downstream of the air channel 8. The heat seal unit is a generally available unit, comprising two bars 11, 12 which are movable with respect to each other to clamp the neck 62 of the bag 7. Both of the bars 11, 12 are coated with a non-stick material, for example Teflon (Trade Mark) to ensure that the molten plastics material of the bag 7 does not adhere to the bars 11, 12. One of the bars 12 includes a heating element which is heated by a current to melt the plastics material and weld the two sides of the neck 62 of the bag 7 together. The seal unit is also able to form a unique identification in the seal to improve the security of the seal. This may be achieved by a unique pattern formed on the bars of the heat seal unit.

One of the bars 11 is pivotally mounted to allow this to be moved out of the way when a bag 7 is to be mounted on the air channel 8. One of the bars 12 is actuated by a motor or solenoid 13 to drive the bars 11, 12 with respect to each other to clamp the neck 62 of the bag 7. This bar 12 is moved between a rest position about 50 mm from the neck 62 of the bag 7 and a seal position by the motor or solenoid 13. A microswitch (not shown) is provided to detect when the bars 11, 12 are clamping the neck 62 of the bag 7, and applies a current to the heater element for a predetermined period, typically 3 seconds, to weld the neck 62 of the bag 7 closed. A detector detects the current flow through the heater elements to confirm that the heat sealing has taken place before access to the package is permitted.

The secure case is locked by an electronic lock 14, including a solenoid or other magnetic element. When the key is actuated to unlock the case, before the lock is released, the heat seal unit is actuated to seal the bag 7. Only then is the case opened to allow access to the bag 7. In this way, the bag 7 is sealed to form a tamper evident package before it can be accessed, giving a high degree of security.

As will be described below, under certain circumstances, in particular where there is a note in the neck 62 of the bag 7 in the region where the bag 7 is to be sealed, it is desirable not to heat seal the bag 7. In this case, the electronic lock 14 will not allow the case to open. In this case, it is necessary for the case to be opened by an over-ride lock which can be actuated only be a person with a high security level, for example a bank employee or a senior supervisor. The over-ride lock may be in the form of a magnetic touch key, for example a Dallas DS19xx Touch Key Memory, or a digital membrane keypad, requiring the entry of a correct code to gain access to the unit. It is possible that different levels of entry code are provided for operators of different seniority. Such a device provides an over-ride lock that is within the casing, and which is not visible externally. Accordingly, there is no external indication of its presence and location. The lock is opened by a high security, unique passive key which interacts with the specific lock to over-ride the normal electronic lock 14 to give access to the secure unit and the unsealed bag 7 contained therein.

As previously described, the end of the air channel 8 remote from the validator 5 includes a sensor to detect the presence and direction of movement on a note within the air channel 8. The sensor may comprise a number of proximity sensors, for example a number of light emitter and detector pairs, arranged in the direction of travel of the note through the air channel 8. The presence of a note can be determined when any of the light detectors does not receive light from its associated light emitter due to a note blocking the light path between them. The direction of movement of a note can be determined by the order in which the light is blocked for the different sensors. For example, where the light is blocked first between the emitter-detector pair nearest the validator 5 and then between the pair nearest the end of the air channel 8 remote from the validator 5, it can be determined that the note is moving from the validator 5 towards the bag 7. When the light is initially blocked between all pairs, and then light is received first by the detector nearest the end of the air channel 8 remote from the validator 5 and then by the detector nearest the validator 5, it is determined that the note is moving in the direction away from the bag 7 and towards the validator 5. This may occur when a note is rejected by the validator 5, or when there is an attempt to remove notes from the secure unit.

The provision of a proximity sensor in the air channel 8 allows the system to confirm that there are no notes in the neck 62 of the bag 7 when the case is unlocked to prevent the bag 7 sealer from sealing the neck 62 of the bag 7, and sealing the note in place. The proximity sensor can also confirm that the notes leaving the validator 5 are received within the bag 7, and can confirm that no notes are jammed within the air channel 8. The system ideally records the receipt of a note within the bag 7 only when it is determined by the sensor that the note has passed through the air channel 8 and into the bag 7, rather than when the note is determined by the validator 5. The reason for this is that it is possible for a note to pass through the validator 5 and remain in the air channel 8. When a subsequent note is inserted into the validator 5, the note may be rejected for a number of reasons. However, the leading edge of the note may contact the note stuck within the air channel 8, and when the note is rejected, this may pull the jammed note from the air channel 8. Therefore, if the content of the bag 7 is determined purely on the output from the validator 5, it will be determined by the system that the note jammed in the air channel 8 and rejected with a following note will be in the bag 7, where as, in fact, the note will not be in the bag 7. By only determining that the note has entered the bag 7 after the note has been determined to have left the air channel 8, the notes in the bag 7 are determined correctly.

A note may be rejected by the validator 5 if the parameters of the note do not fall within the predetermined parameters of an acceptable note. This rejection is a function of the validator 5.

In one example of the present invention, one or more secure units are provided at each sales point to receive payments at the sales point. In this case, the secure unit includes an interface to link the unit to the EPOS system and to a central unit. This allows determination of the content of the tamper evident packages which can save much time in balancing payments at the end of trading.

In an alternative example, a secure unit is provided for cheques in a cash room or other central location in the store. Cheques are processed in the usual manner by the sales assistant, and collected in the till drawer. The cheques are later removed from the till drawer. The cheques are then fed into the secure unit which identifies the cheques. The secure unit includes an identification means instead of the validator as described above. The identification means may be an optical character reader to read information from the cheque. However, it is preferred that the identification means is a bar code reader which reads a specially printed bar code on the cheque. The bar code includes details relating to the cheque and the transaction for which the cheque was tendered such as the amount, transaction time and number, card details etc. This bar code is printed on the reverse of the cheque by the cheque printer provided at the point of sale. The cheque is then fed to the tamper evident package in the unit, which is sealed before it can be removed. In this way, a tamper evident package containing identified cheques is produced. It will be appreciated that this arrangement can be used for other forms of payment such as credit and debit card payments and coupons.

1-26. (canceled)

27. A security unit comprising a secure, lockable housing including an inlet (16) for items to be stored, a gas feed mechanism (8) for conveying items introduced into the inlet (16) of the housing, and a removable package (7) provided within the housing, the removable package (7) including an inlet (62) through which items conveyed by the gas feed mechanism (8) are introduced into the removable package (7) and a gas outlet (61), separate from the inlet, through which gas entering the removable package (7) to convey the items to be stored can escape from the package (7), the unit being arranged so that the inlet (62) of the removable package (7) is sealed in a tamper evident manner before the removable package (7) can be removed from the housing.

28. A security unit according to claim 27, in which the removable package (7) is formed of a plastics material.

29. A security unit according to claim 28, further including a heat seal system (11,12) for heat sealing the removable package (7).

30. A security unit according to claim 29 further comprising a means to detect the sealing of the removable package before allowing access to the removable package.

31. A security unit according to claim 30 in which the means to detect the sealing of the removable package includes a means to detect current flow through the heat seal unit.

32. A security unit according claim 27 further comprising a lock (14) having a delay such that the access to the interior of the housing is prevented for a predetermined period after an attempt is made to open the case.

33. A security unit according to claim 32 a heat seal unit (11,12) that is activated in response to the attempt to open the housing.

34. A security unit according to claim 27 further comprising a sensor associated with the sealing mechanism to verify that there are no items in the position where the removable package is to be sealed.

35. A security unit according to claim 27 further comprising an identification and/or validation means (5) between the inlet of the housing and the removable package (7) for identifying and/or validating items which are introduced to the removable package (7).

36. A security unit according to claim 35, in which the identification and/or validation of the items is stored in a memory.

37. A security unit according to claim 36, in which the removable package (7) is identifiable, and can be associated with the stored identification and/or validation of the items stored in the memory.

38. A security unit according claim 27 in which a sensor (43) detects the presence and/or the correct fitting of a removable package (7), and prevents the use of the unit unless a removable package (7) is present and/or fitted correctly.

39. A secure unit according to claim 27 further comprising gas feed conveying system including a channel through which items are to be conveyed and a pair of gas inlets provided on opposites sides of the channel inclined at an angle to that in which the items are to be conveyed, and through which gas from outside the channel is jetted to produce a gas flow through the channel which entrains an item to convey this through the channel.

40. A package for use in the secure unit according to claim 27 the package comprising a plastics container having an inlet opening for receiving items to be collected, a neck portion extending from the inlet opening, and a number of gas outlets remote from the inlet opening through which gas introduced into the inlet is vented.

41. A package according to claim 40 formed from a plastic material.

42. A package according to claim 40, including fluid outlet holes provided in the wall of the package towards the upper part of the package remote from the inlet to the package.

43. A gas feed conveying system for use in a secure unit the gas feed conveying system comprising a channel (8) through which items are to be conveyed and a pair of gas inlets provided on opposed sides of the channel (8) inclined at an angle to that in which the items are to be conveyed, and through which gas from outside the channel (8) is jetted to produce a gas flow through the channel (8) which entrains an item to convey this through the channel.

44. A gas feed conveying system according to claim 43, in which the gas is air.

45. A gas feed conveying system according to claim 43 in which the channel (8) has a generally oval cross-sectional channel in a plane generally normal to that along which the items are conveyed.

46. A gas feed conveying system according to claim 43 in which the inlets are formed at an angle of about 45 to the direction in which items are conveyed through the channel (8).

47. A gas feed conveying system according to claim 43 in which the inlets are provided at the entrance of the channel (8), and are arranged such that the gas flows from the inlets converge towards the longitudinal central plane of the channel (8).

48. A gas feed conveying system according to claim 43 in which the inlets are formed by providing a hole though a dimple formed in the surface of the channel.

49. A gas feed conveying system according to claim 43 in which the inlets comprise elongate slots extending substantially across the width of the channel.

50. A gas feed conveying system according to claim 43 in which the channel is formed of sheet material, such as metal, and in which the inlets are formed in the flat sheet which is then bent to form the channel.

51. A gas feed conveying system according to claim 43 in which the channel is formed from a plastics material.

52. A gas feed system according to claim 43 in which sensors are provided to detect the movement of items through the channel, and control means are provided to control the flow of gas through the inlets dependent upon the detection of movement of items through the channel.
